(19) United States
(12) Patent Application Publication
(10) Pub. No.: US 2009/0291504 A1
(43) Pub. Date: Nov. 26, 2009

(54) CRE COMPLEMENTATION USING LEUCINE ZIPPER

(76) Inventors: Guoquiang Gu, Nashville, TN (US); Yanwen Xu, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 12/123,860

(22) Filed: May 20, 2008

(51) Int. Cl. C12N 15/74; C12N 9/10; C12N 9/96; C12Q 1/68

(52) U.S. Cl. 435/471; 435/193; 435/188; 435/320.1

(57) ABSTRACT

Cre activity is effectively reconstituted from two modified, inactive Cre fragments, both in cell culture and in transgenic mice. Cre reassociation was facilitated by fusing fragments separately to peptides that can form a tight anti-parallel leucine zipper. The co-expressed Cre fusion fragments showed substantial activity in cultured cells. Also disclosed are in vivo techniques for increasing the cell-specificity of gene manipulation, where expression in transgenic mice using individual promoters for each Cre fragment effectively reconstituted Cre activity, and the activation of LoxP recombination in the co-expressing cells.

This application claims benefit of priority to U.S. Provisional Application Ser. No. 60/639,241, filed May 21, 2007, the entire contents of which are hereby incorporated by reference

This invention was made with government support under grant number 1RO1 DK065949-01A1 awarded by the National Institutes of Health. The government has certain rights in the invention.

### BACKGROUND OF THE INVENTION

1. Field of the Invention

The present invention relates generally to the fields of cellular and molecular biology. More particularly, it concerns Cre fusion proteins comprising leucine zipper motifs to reconstitute Cre activity in a cell based on α-β complementation. The technology may be used in a variety of contexts, and in particular, may be used to mark stem cells early in development, and in adult animals under normal physiological and regenerative conditions.

2. Description of Related Art

The Cre/LoxP system utilizes P1 bacteriophage Cre recombinase to catalyze recombination between tandem LoxP DNA sequences (Abremski and Hoess, 1984; Abremski and Hoess, 1985). This system has been widely used in multiple organisms, including yeast (Sauer, 1987), plants (Bayley et al., 1992; Gilbertson, 2003; Qin et al., 1994; Radhakrishnan and Srivastava, 2005), and animals (Lakso et al., 1992; LePage and Conlon, 2006; Metzger and Chambon, 2001; Porret et al., 2006; Rajewsky et al., 1996; Sauer, 2002; Sauer and Henderson, 1988). The Cre/LoxP technology is particularly useful for mammalian genetics, because it allows the analyses of essential genes in specific organs by gene inactivation (Lakso et al., 1992; LePage and Conlon, 2006; Metzger and Chambon, 2001; Porret et al., 2006; Rajewsky et al., 1996; Rossant and Nagy, 1995; Sauer, 2002; Sauer and Henderson, 1988) or controlled ectopic gene expression (Branda and Dymecki, 2004; Lewandoski, 2001).

When combined with visible marker proteins, Cre-LoxP-based gene activation allows for cell marking and cell lineage analyses in living animals (Branda and Dymecki, 2004). Specific gene promoters are usually utilized to drive Cre expression in desired tissues. Thus, the promoter specificity limits where Cre can be expressed. To this end, most available promoters drive gene expression in multiple cell types. This deficiency has greatly limited the ability to manipulate genes within specific cells, such as stem cells that can only be identified by their expression of several molecular markers (Case et al., 2005; Howell et al., 2003; Kiel et al., 2005). An approach that introduces Cre exclusively to cells that express more than one protein marker would facilitate our understanding of the function and fates of specific cells in vivo.

Active protein can be reconstituted from peptide fragments of corresponding molecules. For some proteins, fragmented peptides can directly re-associate to restore activity (Remy and Michnick, 2004; Richards and Vithayathil, 1959; Shiba and Schimmel, 1992). In other scenarios, assisted protein reconstitution is required. In this latter case, protein can be cleaved to two inactive fragments. Each fragment was then fused to one of a pair of interacting protein motifs respectively. The interacting motifs could bring the protein fragments to proximity to facilitate efficient reassembly (Chelur and Chalfie, 2007; Johnsson and Varshavsky, 1994; Magliery et al., 2005; Pelletier et al., 1999; Pelletier et al., 1998; Remy and Michnick, 2004; Rossi et al., 1997; Zhang et al., 2004). Both above schemes have been explored for Cre activity reconstitution (Casanova et al., 2003; Jullien et al., 2003). In one report, Cre was cleaved into two inactive halves and expressed in same cells. About 10-15% Cre activity was reportedly restored (Casanova et al., 2003; Shimshek et al., 2002). In the other case, two inactive Cre moieties were connected with two interacting proteins, FK506 binding protein (FKBP12) and FKBP12-rapmycin-associated-Protein (FRP). Because the interaction of FKBP12 and FRP is FK506 dependent, Cre activity could be restored only when both moieties and FK506 were present (Jullien et al., 2003; Kellendonk et al., 1996). This method restores about 4% Cre activity (Jullien et al., 2003). The usefulness of these two systems in animal models has not been reported, but the low activity level of the reconstituted enzyme clearly renders these approaches of limited applicability.

### SUMMARY OF THE INVENTION

Thus, in accordance with the present invention, there is provided an active Cre recombinase comprising (i) an N-terminal segment of Cre fused to a first leucine zipper peptide, wherein said first leucine zipper peptide is fused to the C-terminal end of said N-terminal segment; and (ii) a C-terminal segment of Cre fused to a second leucine zipper peptide, wherein said second leucine zipper peptide is fused to the N-terminal end of said C-terminal segment. The first and/or second leucine zipper peptide may be between 20 and 40 residues in length, and in particular, the first leucine zipper may comprise the sequence GALKKELQANKKELAQLKWELQALKKELAQ (SEQ ID NO:1), and the second leucine zipper may comprise the sequence EQLEKKLQALEKKLAQLEWKNQALEKKLAQG (SEQ ID NO:2). The N-terminal segment may comprise residue 1 to about residue 190 of native Cre recombinase. The C-terminal segment may comprise from about residue 191 to residue 320 of native Cre recombinase. The N-terminal and/or C-terminal segments may further comprise a nuclear localization signal, for example, an SV40 nuclear localization signal. In particular, the nuclear localization signal may be located at the N-terminus of said N-terminal segment; alternatively, the nuclear localization signal may be located at the N-terminus of said second leucine zipper peptide.

In another embodiment, there is provided an N-terminal segment of Cre fused to a leucine zipper peptide, wherein said leucine zipper peptide is fused to the C-terminal end of said N-terminal segment. The N-terminal segment may further comprise a nuclear localization signal. In yet another embodiment, there is provided a C-terminal segment of Cre fused to a leucine zipper peptide, wherein said leucine zipper peptide is fused to the N-terminal end of said C-terminal segment. The C-terminal segment may further comprise a nuclear localization signal.

In a further embodiment, there is provided an expression construct encoding (i) a fusion protein comprising an N-terminal segment of Cre fused to a leucine zipper peptide, wherein said leucine zipper peptide is fused to the C-terminal end of said N-terminal segment, and (ii) a promoter. The expression construct may further encode a nuclear localization signal a part of said fusion protein.

In yet a further embodiment, there is provided a (i) a fusion protein comprising a C-terminal segment of Cre fused to a leucine zipper peptide, wherein said leucine zipper peptide is fused to the N-terminal end of said C-terminal segment, and (ii) a promoter. The expression construct may further encode a nuclear localization signal as part of said fusion protein.

Another embodiment comprises a kit comprising, in separate containers (a) an expression construct encoding (i) a first fusion protein comprising an N-terminal segment of Cre fused to a first leucine zipper peptide, wherein said first leucine zipper peptide is fused to the C-terminal end of said N-terminal segment, and (ii) a promoter that directs expression of said first fusion protein; and (b) an expression construct encoding (i) a second fusion protein comprising a C-terminal segment of Cre fused to a second leucine zipper peptide, wherein said second leucine zipper peptide is fused to the N-terminal end of said C-terminal segment, and (ii) a promoter that directs expression of said second fusion protein. The expression construct may further encode a nuclear localization signal as part of first and/or second fusion proteins.

In still yet another embodiment, there is provided a method for introducing Cre recombinase activity into a cell lacking cre recombinase activity comprising (a) a first expression cassette encoding (i) a first fusion protein comprising an N-terminal segment of Cre fused to a first leucine zipper peptide, wherein said first leucine zipper peptide is fused to the C-terminal end of said N-terminal segment, and (ii) a promoter that directs expression of said first fusion protein; and (b) a second expression cassette encoding (i) a first fusion protein comprising a C-terminal segment of Cre fused to a second leucine zipper peptide, wherein said second leucine zipper peptide is fused to the N-terminal end of said C-terminal segment and (ii) a promoter that directs expression of said second fusion protein. The first and/or second leucine zipper peptide may be between 20 and 40 residues in length, for example, where the first leucine zipper comprises the sequence GALKKELQANKKELAQLKWELQALKKELAQ (SEQ ID NO:1) and the second leucine zipper comprises the sequence EQLEKKLQALEKKLAQLEWKNQALEKKLAQG (SEQ ID NO:2).

The N-terminal segment may comprise residue 1 to about residue 190 of native Cre recombinase. The C-terminal segment comprises from about residue 191 to residue 320 of native Cre recombinase. The N-terminal and/or C-terminal segments may further comprise a nuclear localization signal, for example, an SV40 nuclear localization signal. In particular, the nuclear localization signal may be located at the N-terminus of said N-terminal segment; alternatively, the nuclear localization signal may be located at the N-terminus of said second leucine zipper peptide.

Introducing may comprise non-viral transfer, such as contacting a lipid vehicle comprising said expression cassettes with said cell, or with pronuclear DNA injection. Introducing may also comprise viral transfer, such as contacting said cell with a first replication deficient vector encoding said first expression cassette; and a second replication deficient vector encoding said second first expression cassette. Alternatively, viral transfer may comprise contacting said cell with a replication deficient viral vector encoding said first expression cassette and said second first expression cassette. Introducing comprises pronuclear injection.

The cell may be stem cell, for example, first promoter is a promoter for a first stem cell marker, and/or said second promoter is a promoter for a second stem cell marker. The method may further comprise introducing a marker segment into said cell, said marker segment comprising an expression cassette comprising two loxP sites and a detectable or selectable marker gene under the control of a third promoter active in said cell, said expression cassette flanked by said loxP sites. The cell may also be a zygote, a pluripotent cell, a totipotent cell or a gene therapy target cell.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. A diagram of the half-Cre molecules and the interacting peptide sequences. (FIG. 1A), Cre was cleaved into two molecules between two glycine residues (amino acid residues 190-191, as numbered in X03453). The N-terminal half was fused with one of a pair peptides that form a leucine zipper (N-peptide), whereas the C-terminal half was fused with the other peptide (C-peptide). (FIG. 1B), the C— and N-peptide sequences.

FIGS. 2A-E. The scheme for Cre activity assay. (FIG. 2A), the structure of the Cre reporter. EGFP can be expressed only after Cre-mediated excision of the stop signal. (FIG. 2B), one example of Cre activity assays. (FIG. 2B1), GFP-expressing cells, indicating detectable Cre activity. (FIG. 2B3), mCherry-expressing cells indicating transfected cells. (FIG. 2B2), merge of B1 and B2. (FIG. 2C), one example of flow cytometry analyses to reveal the percentage of red cells that are green. Shown are only cells that express mCherry (in this example, 13.4% red cells were green). (FIG. 2D), the GFP+/mCherry+ cells vs. the amount of Cre-expressing plasmid (the X-axis is the amount of plasmid used for one well of 6-well dishes). (FIG. 2E), relative Cre activity reconstituted from each Cre fragment combination.

FIGS. 3A-B. Transgene structures and a mouse cross scheme. (FIG. 3A), DNA constructs that utilize a Pdx1 promoter to drive the expression of nCre and nlcCre. (FIG. 3B), an example of mouse cross scheme to produce animals of desired genotype.

FIGS. 4A-F. Reconstituted Cre activates reporter gene expression in transgenic mouse. Green fluorescence indicates YFP+ cells that have undergone Cre-mediated recombination. (FIGS. 4A-C), E13.0. (FIGS. 4D-F), E17.5. (FIG. 4A), a pancreas of an nCretg; R26R-EYFP (=R26YFP) animal. The green channel in this picture was enhanced to visualize the lobular pancreatic structure in the dorsal pancreas. (FIGS. 4B-E), pancreata of nCretg; nlcCretg;R26RYFP animals. (FIG. 4F), a ventral pancreas of an nCretg; nlcCretg; R26YFP/R26YFP animal. Also note the presence of YFP+ cells in the duodenum. dp, dorsal pancreas; vp, ventral pancreas; du, duodenum. Bar=40 μm.

FIGS. 5A-I. Cre activity can be reconstituted in all pancreatic progenitors. Green fluorescence indicates YFP+ cells. Shown are pancreatic regions only. Red fluorescence indicates the expression of pancreatic markers, as marked in each panel. (FIGS. 5A-F), neonatal pancreas. (FIGS. 5G-I), 2 month-old adult pancreas. Yellow arrows point to double positive cells. amy, amylase; ins, insulin; SS, somatostatin; DBA, Dolichos-Biflorus agglutinin; glc, glucagon; PP, pancreatic polypeptide. Bar=20 μm.

### DESCRIPTION OF ILLUSTRATIVE EMBODIMENTS

### I. THE PRESENT INVENTION

The inventors attempted to reconstitute Cre in mouse cells that express two protein markers. Because the inventors could obtain less than 2% Cre activity using the published Cre fragment complementation (Casanova et al., 2003), the inventors utilized assisted-Cre reassembly for this purpose. Cre was cleaved into two inactive molecules and each was fused to one of two peptides that could form anti-parallel leucine zippers (Wilson et al., 2004). This leucine zipper was artificially designed and has been reported to effectively assist protein reconstitution in vitro and in vivo, and these peptides do not seem to interfere with normal cellular functions (Pelletier et al., 1999; Wilson et al., 2004; Zhang et al., 2004). Results showed that about 30% Cre activity could be reconstituted from these two inactive Cre fragments in tissue culture, an 8-fold improvement over previously published methods (Casanova et al., 2003; Jullien et al., 2003). When expressed in the pancreatic tissue of transgenic mouse from individual promoters, the inactive Cre fragments effectively induce LoxP-based recombination. This approach opens the possibility to study gene function or perform lineage labeling in cells that express dual protein markers.

### II. CELL TYPES

In accordance with the present invention, a variety of different cells may be targeted with the Cre recombinase complexes of the present invention. There is little constraint on the type of cell that can be utilized other than (a) the absence of Cre activity in that cell and (b) the amenability of the cell to receive exogenous nucleic acids. Various particular target cells are discussed below.

1. Stem Cells

Stem cells differ from other kinds of cells in the body. All stem cells, regardless of their source, have three general properties: they are capable of dividing and renewing themselves for long periods; they are unspecialized; and a single stem cell can give rise to multiple specialized cell types. One of the fundamental properties of a stem cell is that it does not have any tissue-specific structures that allow it to perform specialized functions. A stem cell cannot work with its neighbors to pump blood through the body (like a heart muscle cell); it cannot carry molecules of oxygen through the bloodstream (like a red blood cell); and it cannot fire electrochemical signals to other cells that allow the body to move or speak (like a nerve cell). However, unspecialized stem cells can give rise to specialized cells, including heart muscle cells, blood cells, or nerve cells.

Unlike muscle cells, blood cells, or nerve cells, which do not normally replicate themselves, stem cells may replicate many times. When cells replicate themselves many times over it is called proliferation. A starting population of stem cells that proliferates for many months in the laboratory can yield millions of cells. If the resulting cells continue to be unspecialized, like the parent stem cells, the cells are said to be capable of long-term self-renewal.

The specific factors and conditions that allow stem cells to remain unspecialized are of great interest to scientists. It has taken scientists many years of trial and error to learn to grow stem cells in the laboratory without them spontaneously differentiating into specific cell types. For example, it took 20 years to learn how to grow human embryonic stem cells in the laboratory following the development of conditions for growing mouse stem cells. Therefore, an important area of research is understanding the signals in a mature organism that cause a stem cell population to proliferate and remain unspecialized until the cells are needed for repair of a specific tissue. Such information is critical for scientists to be able to grow large numbers of unspecialized stem cells in the laboratory for further experimentation.

When unspecialized stem cells give rise to specialized cells, the process is called differentiation. Scientists are just beginning to understand the signals inside and outside cells that trigger stem cell differentiation. The internal signals are controlled by a cell's genes, which are interspersed across long strands of DNA, and carry coded instructions for all the structures and functions of a cell. The external signals for cell differentiation include chemicals secreted by other cells, physical contact with neighboring cells, and certain molecules in the microenvironment.

Embryonic stem cells, as their name suggests, are derived from embryos. Specifically, embryonic stem cells are derived from embryos that develop from eggs that have been fertilized in vitro, i.e., in an in vitro fertilization clinic, and then donated for research purposes with informed consent of the donors. They are not derived from eggs fertilized in a woman's body. The embryos from which human embryonic stem cells are derived are typically four or five days old and are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel.

Adult stem cells typically generate the cell types of the tissue in which they reside. A blood-forming adult stem cell in the bone marrow, for example, normally gives rise to the many types of blood cells such as red blood cells, white blood cells and platelets. Until recently, it had been thought that a blood-forming cell in the bone marrow, which is called a hematopoietic stem cell, could not give rise to the cells of a very different tissue, such as nerve cells in the brain. However, a number of experiments over the last several years have raised the possibility that stem cells from one tissue may be able to give rise to cell types of a completely different tissue, a phenomenon known as plasticity. Examples of such plasticity include blood cells becoming neurons, liver cells that can be made to produce insulin, and hematopoietic stem cells that can develop into heart muscle. Therefore, exploring the possibility of using adult stem cells for cell-based therapies has become a very active area of investigation by researchers.

2. Progenitor Cells

A stem cell can divide to produce two daughter cells. Under normal conditions, one daughter cell maintains the properties of the stem cell (called stem cell renewal). The other daughter will give rise to specialized (or terminally differentiated) and functional cells. Yet this later daughter cell usually does not directly differentiate into a single functional cell. Instead, it divides for several rounds so that multiple functional cells arise from a single immediate daughter of the stem cell. This “immediate daughter” cell is referred to as a “progenitor cell.” Progenitor cells differ from either stem cells or specialized cells. Like stem cells, progenitor cells can proliferate and give rise to, but do not perform functions of, specialized cell. Unlike stem cells, a single progenitor cell cannot give rise to multiple mature cell types. Progenitor cells cannot perform functions, as specialized cells, such as working with its neighbors to pump blood through the body (like a heart muscle cell); carring molecules of oxygen through the bloodstream (like a red blood cell); and firing electrochemical signals to other cells that allow the body to move or speak (like a nerve cell).

The molecular programs governing the proliferation and differentiation of progenitor cells differ from that of stem cells. By performing gene activity manipulation in specific progenitor cells, scientists have revealed, and will continue to reveal, what molecules make a stem cell differ from a stem cell, what molecules make a progenitor divide, and what molecules make a progenitor differentiate to a specialized cell. These lines of information are critical in understanding how each tissue or cell type within an animal communicates with each other so that the right number of cells can be produced for normal function, yet preventing cancerous, uncontrollable cell proliferation. These studies are also essential in directing embryonic stem cell differentiation toward desired mature, functional cell types that could be utilized for cell based therapy.

3. Specialized/Differentiated Cells

Specialized/differentiated cells are the functional units in the body. These cells can be classified as two major classes according to their life span. Some specialized cell types, including most nerve cells, live as long as the individual wherein these cells reside. These cells do not divide and adult individuals contain not detectable progenitors for these cells. Specialized mechanisms are employed to protect these cells from damages or stresses and debilitating diseases, such as Alzheimer's' disease, occur when these protecting mechanisms go awry. The other cell type has limited life span, i.e. they only live a limited period of time and compensatory mechanisms are required to replenish lost cells. Two general mechanisms are utilized to produce the lost cells. First, some specialized cells, including blood cells, some nerve cells, gut cell, and epidermal cells, could not proliferate. When these cells die, proliferation/differentiation of stem or progenitor cells maintain these cells at a steady number. Second, some other specialized functional cells, such as liver cells or pancreatic cells, can divide. In this case, proliferation of existing specialized cells replenishes lost cells caused by damage or normal cell death.

In all above scenarios, scientists are exploring the genes and gene networks that control the balance of cell death and proliferation. These lines of research are critical for understanding the mechanisms how mature tissues communicate with each other or with their stem cell/progenitors to maintain a normal organ homeostasis and function.

### III. EXPRESSION CONSTRUCTS

Throughout this application, the term “expression construct” is meant to include any type of genetic construct containing a nucleic acid coding for a gene product in which part or all of the nucleic acid encoding sequence is capable of being transcribed. In such embodiments, the nucleic acid encoding the gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

1. Tissue- and Cell-Specific Promoters

Of particular interest are tissue- and cell-specific promoters. For example, muscle specific promoters, and more particularly, cardiac specific promoters, are useful in preparing immortalized cardiac cell lines. These include the myosin light chain-2 promoter (Franz et al., 1994; Kelly et al., 1995), the α actin promoter (Moss et al., 1996), the troponin 1 promoter (Bhavsar et al., 1996); the Na+/Ca2+ exchanger promoter (Barnes et al., 1997), the dystrophin promoter (Kimura et al., 1997), the creatine kinase promoter (Ritchie, 1996), the α7 integrin promoter (Ziober & Kramer, 1996), the brain natriuretic peptide promoter (LaPointe et al., 1996), the αB-crystallin/small heat shock protein promoter (Gopal-Srivastava, 1995), and α myosin heavy chain promoter (Yamauchi-Takihara et al., 1989) and the ANF promoter.

Stem cell promoters also are contemplated for use according to the present invention. Examples for this class of promoters are plenty. For examples, a combination of CD34 (Civin et al., 1984), cD133 (Uchida et al., 2000), ScaI (Ishikawa et al., 2006) and/or cKit promoters (Hunte et al., 1998) marks blood stem cells. A combination of Nestin (Frederiksen et al., 1988), PSA-NCAM (Theodosis et al., 1994), and/or P75 neurotrophin receptor (Theodosis et al., 1994) promoters marks neural stem cells. A combination of SP-C and CCA promoters marks lung stem cells (Kim et al., 2005).

Progenitor or and mature cell-specific promoters could also be utilized. Combination of these markers helps to define specific cell populations. For example, cytokeratin 18 promoter (Gunther et al., 1995), albumin promoter (Liu et al., 1988), insulin promoter (Herrera, 2000), glucagon promoter (Herrera, 2000), Pancreatic polypeptide promoter (Herrera, 2000), Pdx1 promoter (Wu et al., 1997), and the Ptf1a promoter (Kawaguchi et al., 2002).

2. Nuclear Localization Signals

A nuclear localizing sequence (NLS) is an amino acid sequence which acts like a “tag” on the exposed surface of a protein. This sequence is used to confine the protein to the cell nucleus through the Nuclear Pore Complex and to direct a newly synthesized protein into the nucleus via its recognition by cytosolic nuclear transport receptors. Typically, this signal consists of a few short sequences of positively charged lysines or arginines. Different nuclear localized proteins may share the same NLS.

Genetically, the NLS results from transcription of a nuclear localizing sequence. Cellular processes and protein function may be studied by adding a known NLS sequence to a gene, confining the chimeric protein product to the nucleus. A NLS has the opposite function of a nuclear export signal, which confines proteins to the cytosolic face of the nuclear membrane. The first NLS was discovered in the Simian Virus 40. It has the squence(NH2)-Pro-Pro-Lys-Lys-Lys-Arg-Lys-Val-(COOH) (SEQ ID NO:3) and is very often used in cell biology to transfer proteins to the nucleus of a cell.

A protein translated with a NLS will bind strongly to importin (a.k.a. karyopherin), and together, the complex will move through the nuclear pore. At this point, Ran-GTP will bind to the importin-protein complex, and its binding will cause the importin to lose affinity for the protein. The protein is released, and now the Ran-GTP/importin complex will move back out of the nucleus through the nuclear pore. A GTPase activating protein (GAP) in the cytoplasm hydrolyzes the Ran-GTP to GDP, and this causes a conformational change in Ran, ultimately reducing its affinity for importin. Importin is released and Ran-GDP is recycled back to the nucleus where guanine exchange factor (GEF) exchanges its GDP back for GTP.

3. IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). According to the present invention, two nucleic acids segments encoding the two portions of Cre may be incorporated into the same expression construct, thereby permitting expression of both with a single construct and a single selectable marker.

IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

### IV. CRE-LOX

Cre is a 38 kDa recombinase protein from bacteriophage P1 which mediates intramolecular (excisive or inversional) and intermolecular (integrative) site specific recombination between loxP sites (see Sauer, 1993). A loxP site (locus of X-ing over) consists of two 13 bp inverted repeats separated by an 8 bp asymmetric spacer region. One cre gene can be isolated from bacteriophage P1 by methods known in the art, for instance, as disclosed by Abremski et al. (1983), the entire disclosure of which is incorporated herein by reference. U.S. Pat. No. 4,959,317, incorporated by reference, describes the basic Cre-Lox system.

One molecule of Cre binds per inverted repeat, or two Cre molecules line up at one loxP site. The recombination occurs in the asymmetric spacer region. Those 8 bases are also responsible for the directionality of the site. Two loxP sequences in opposite orientation to each other invert the intervening piece of DNA, two sites in direct orientation dictate excision of the intervening DNA between the sites leaving one loxP site behind. This precise removal of DNA can be used to activate or eliminate a transgene.

Lox sites are nucleotide sequences at which the gene product of the Cre recombinase can catalyze a site-specific recombination. A LoxP site is a 34 base pair nucleotide sequence which can be isolated from bacteriophage P1 by methods known in the art. One method for isolating a LoxP site from bacteriophage P1 is disclosed by Hoess et al. (1982), the entire disclosure of which is hereby incorporated herein by reference. As stated above, the LoxP site consists of two 13 base pair inverted repeats separated by an 8 base pair spacer region. The nucleotide sequences of the insert repeats and the spacer region of LoxP are as follows:

| | |
|---|---|
| ATAACTTCGTATA ATGTATGC TATACGAAGTTAT | (SEQ ID NO:4) |

Other suitable lox sites include LoxB, LoxL and LoxR sites which are nucleotide sequences isolated from E. coli. These sequences are disclosed and described by Hoess et al. (1982), the entire disclosure of which is hereby incorporated herein by reference. Preferably, the lox site is LoxP or LoxC2. The nucleotide sequences of the insert repeats and the spacer region of LoxC2 are as follows:

| | |
|---|---|
| ACAACTTCGTATA ATGTATGC TATACGAAGTTAT | (SEQ ID NO:5) |

Johnson et al., in PCT Application No. WO 93/19172, the entire disclosure of which is hereby incorporated herein by reference, describes phage vectors in which the VH genes are flanked by two loxP sites, one of which is a mutant loxP site (loxP 511) with the G at the seventh position in the spacer region of loxP replaced with an A, which prevents recombination within the vector from merely excising the VH genes. However, two loxP 511 sites can recombine via Cre-mediated recombination and, therefore, can be recombined selectively in the presence of one or more wild-type lox sites. The nucleotide sequences of the insert repeats and the spacer region of loxP 511 as follows:

| | |
|---|---|
| ATAACTTCGTATA ATGTATAC TATACGAAGTTAT | (SEQ ID NO:6) |

Lox sites can also be produced by a variety of synthetic techniques which are known in the art. For example, synthetic techniques for producing lox sites are disclosed by Ito et al. (1982) and Ogilvie et al. (1981), the entire disclosures of which are hereby incorporated herein by reference.

### V. LEUCINE ZIPPERS

The leucine zipper is a type of structural motif found in parallel or antiparallel coiled coils. It is a common dimerization domain found in some proteins involved in regulating gene expression. The main feature of the leucine zipper domain is the predominance of the common amino acid leucine at the d position of the heptad repeat. Leucine zippers were first identified by sequence alignment of certain transcription factors which identified a common pattern of leucines every seven amino acids. These leucines were later shown to form the hydrophobic core of a coiled coil. Each half of a leucine zipper consists of a short α with a leucine residue at every seventh position. The standard 3.6 residues per turn α-helix structure changes slightly to become a 3.5 residues per turn alpha-helix. Known also as the heptat repeat, one leucine comes in direct contact with another leucine on the other strand every second turn.

Examples of leucine zippers can be found Saccharomyces cerevisiae (GCN4, CD-Gal4, PPR1, PUT3, HAP1), Schizosaccharomyces pombe (PAP1), Homo sapiens (C-fos, C-jun, C-myc, Max, Aft4, C/ebpβ, cAMP-dependent transcription factor ATF-2), Rattus norvegicus (C/ebpα) and Mus musculus (Creb, C/ebpβ), Sox family proteins, and hundreds of other human or mouse proteins encoded in their genome (Gray et al., 2004; Wiemann et al., 2001).

### VI. DELIVERY OF NUCLEIC ACIDS

In accordance with the present invention, nucleic acids are delivered to cells, thereby permitting expression of Cre recombinase N— and C-terminal fragments, which interact by virtue of leucine zippers fused thereto. There are two generally types of gene transfer—viral and non-viral. Typically, the same type of gene transfer will be utilized for both nucleic acids encoding both fragments, although different methods may be utilized. Each of these are described below.

1. DNA Delivery Using Viral Vectors

The ability of certain viruses to infect cells and/or enter cells via receptor-mediated endocytosis, and/or to integrate into host cell genome and/or express viral genes stably and/or efficiently have made them attractive candidates for the transfer of foreign genes into mammalian cells. Although some viruses that can accept foreign genetic material are limited in the number of nucleotides they can accommodate or in the range of cells they infect, viruses have been generally successful in effecting gene expression. Different types of viral vectors, and techniques for preparing such, are well known in the art.

A. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a coding region that has been inserted therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

Recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 and/or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, and about 15% of the total length of the vector. More than 80% of the adenovirus viral genome remains in the vector backbone.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus et al., 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing primary cells.

B. AAV Vectors

Adeno-associated virus (AAV) is an attractive vector system for use in the cell transduction of the present invention as it has a high frequency of integration and it can infect nondividing cells, thus making it useful for delivery of genes into cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in various diseases (Flotte et al., 1992; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus or a member of the herpesvirus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

C. Retroviral Vectors

Retroviruses have promise as gene delivery vectors due to their ability to integrate their genes into the host genome, transferring a large amount of foreign genetic material, infecting a broad spectrum of species or cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, cells bearing the erythropoietin (EPO) receptor. This was achieved by inserting a portion of the EPO sequence into an envelope protein to create a chimeric protein with a new binding specificity.

D. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus or herpes simplex virus may be employed. They offer several attractive features for various cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

E. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin (Roux et al., 1989). Using antibodies against major histocompatibility complex class I or class II antigens, they demonstrated the infection of a variety of cells that bore those surface antigens with an ecotropic virus in vitro (Roux et al., 1989).

2. Non-Viral Transformation

Suitable methods for non-viral nucleic acid delivery for transformation of a cell for use with the current invention are believed to include virtually any method by which a nucleic acid (e.g., DNA) as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

A. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of DNA used may vary upon the nature of the antigen as well as the organelle, cell, tissue or organism used

B. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

C. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

D. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

E. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

F. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

G. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

H. Microprojectile Bombardment

Microprojectile bombardment techniques can be used to introduce a nucleic acid into a cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

I. Pronuclear Injection

Microinjection into the pronucleus (Pronuclear Injection, PNI) has been the most used technique for transgenic animal production. Microinjection involves the mechanical introduction of a purified double stranded DNA sequence into the pronucleus of a fertilized mammalian oocyte leading to the integration of the sequence (transgene) within the existing genetic sequence. This integration means that the animal is born with a copy of the new sequence in every cell, making it an excellent method for studying mammalian growth and pathology. See Brinster et al. (1985); Gordon et al. (1980); Jaenisch (1988).

### VII. TRANSGENIC ANIMALS

Transgenic non-human animals (e.g., mammals) of the invention can be of a variety of species including murine (rodents; e.g., mice, rats), avian (chicken, turkey, fowl), bovine (beef, cow, cattle), ovine (lamb, sheep, goats), porcine (pig, swine), and piscine (fish). In a particular embodiment, the transgenic animal is a rodent, such as a mouse or a rat. Detailed methods for generating non-human transgenic animals are described herein. Transgenic gene constructs can be introduced into the germ line of an animal to make a transgenic mammal. For example, one or several copies of the construct may be incorporated into the genome of a mammalian embryo by standard transgenic techniques.

According to the present invention, expression construction comprising Cre recombinase fusion proteins may be introduced into embryonic cells. Embryonic target cells at various developmental stages can be used to receive these constructs. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

Introduction of the constructs into the embryo can be accomplished by any means known in the art such as, for example, microinjection, electroporation, or lipofection. For example, the constructs can be introduced into a mammal by microinjection of the construct into the pronuclei of the fertilized mammalian egg(s) to cause one or more copies of the construct to be retained in the cells of the developing mammal(s). Following introduction of the construct into the fertilized egg, the egg may be incubated in vitro for varying amounts of time, or reimplanted into the surrogate host, or both. Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces. In vitro incubation to maturity is within the scope of this invention. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

Transgenic offspring of the surrogate host may be screened for the presence and/or expression of a transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and sperm obtained from the transgenic animal. Where in vitro fertilization is used, the fertilized embryo may be implanted into a surrogate host or incubated in vitro, or both. Using either method, the progeny may be evaluated for the presence of the transgene using methods described above, or other appropriate methods.

Retroviral infection can also be used to introduce transgene into a non-human animal. The developing non-human embryo can be cultured in vitro to the blastocyst stage. During this time, the blastomeres can be targets for retroviral infection (Jaenich, 1986). Efficient infection of the blastomeres is obtained by enzymatic treatment to remove the zona pellucida (Manipulating the Mouse Embryo, Hogan et al. eds., 1986). The viral vector system used to introduce the transgene is typically a replication-defective retrovirus carrying the transgene (Jahner et al., 1985; Van der Putten et al., 1985). Transfection is easily and efficiently obtained by culturing the blastomeres on a monolayer of virus-producing cells (Van der Putten, 1985; Stewart et al., 1987). Alternatively, infection can be performed at a later stage. Virus or virus-producing cells can be injected into the blastocoele (Jahner et al., 1982). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. 1982).

A third type of target cell for transgene introduction is the embryonal stem cell (ES). ES cells are obtained from pre-implantation embryos cultured in vitro and fused with embryos (Evans et al., 1981; Bradley et al., 1984; Gossler et al., 1986; Robertson et al., 1986). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch (1988).

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

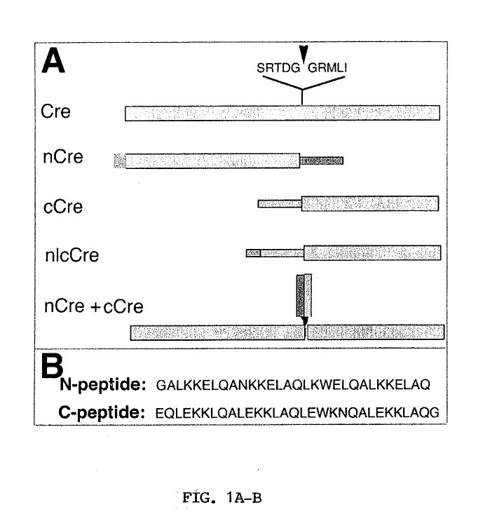

DNA constructs and transgenic mouse production. For fusion of leucine zipper-forming peptides with Cre moieties (FIGS. 1A-B), overlapping DNA oligos were synthesized and PCR-amplified with Cre protein (with a nuclear localization signal (NLS) present in Cre's n-terminus) coding sequence as template (Gu et al., 2002). The N-terminal half of Cre was fused with N-peptide at Cre C-terminus to produce nCre (with a NLS at its N-terminus, FIGS. 1A-B). The oligos utilized were: X5, Nz1, Nz2, Nz3, and nZ4 (Table 1). The C-terminal half of Cre was fused with C-peptide at Cre n-terminus to produce cCre (FIGS. 1A-B). The DNA oligos used were: N3, Cz1, cZ2, cZ3, and cZ4 (Table 1). To add an extra nuclear localization signal to cCre (to produce nlcCre), the inventors utilized the following oligos: Nlc, N3, Cz1, cZ2, cZ3, and, cZ4 (Table 1). PCR-fragments were cloned into pBluescriptKSII vector to produce pYW 415, pYW429, pYW418 respectively. The Xho I-Not I fragments from these constructs were ligated into the corresponding sites of the pCIG-expression vector, containing the CMV-chicken-β actin promoter to drive gene expression, to produce pYW427, pYW443, and pYW425 (Xu et al., 2006). For CMV-stop-GFP, an EcoR I-Spe I fragment (contains a Poly A signal) from pBS302 (Sauer, 1993) was ligated into the EcoR I-Spe I sites of pGreenlatern-1 to produce YW421 (Gu et al., 2004). As control for Cre activity assay, the full length Cre with an NLS at its N-terminus was PCR amplified and inserted into the Xho I-Not I sites of the pCIG vector to produce YW482. The oligos utilized were: fc1 and fc2 (Table 1). Note all reading frames contain an idealized “Kozak sequence” CCACC before ATG. To amplify the α and β fragments reported in (Casanova et al., 2003), DNA oligos (X5+T5) and (N3+T3) were utilized. The pCIG vector was utilized to drive the expression of these fragments as well.

For transgene constructs, PCR-amplified SV40 polyA sequences from pGreenlatern1 were inserted into the SmaI site of pBluescript KSII, producing pGD103 (oligonucleotides utilized: pA1, pA2; Table 1). The Xho I-Not I (filled-in)-digested nCre or nlcCre fragments were inserted into the XhoI-EcoRV site of pGD103, producing YW452 and YW451 respectively. Finally, Xho I (filled-in)-Not I fragments from YW452 and YW451 were inserted downstream of the murine Pdx1 promoter (Sma I/Not I-restricted plasmid #571; gift from C. Wright). Inserts were released with Sal I-Not I for transgenic animal production in the Shared ES Cell/Transgenic Animal Resources in Vanderbilt Medical Center. nCretg and nlcCretg genotyping was with ng1 and cga oligonucleotides (Table 1). R26R-EYFP (R26YFP) and Z/AP reporter animals genotyping, and alkaline phosphatase detection was by published methods (Gu et al., 2002; Srinivas et al., 2001). All mouse care, handling, and crosses followed IACUC protocol M/03/354 (Gu), approved by the Animal Welfare Committee of Vanderbilt Medical Center.

Cre activity assay. A reporter plasmid (YW421) expressing GFP Cre-dependently was used to assay Cre activity. Reporter YW421, plus cCre or nCre plasmids (or both), and mCherry-producing plasmid (Shu et al., 2006) were co-transfected into HK293 cells. After 14-16 hours, transfected cell were analyzed by flow cytometry. The percentage of red cells that express GFP were plotted against the Cre plasmid(s) concentration. For most transfections, 0.2 μg YW421, 0.1 μg mCherry, and 0.1-2 ng Cre-expressing plasmids were utilized for each well of 12-well dishes. With bigger wells, the plasmid amount was scaled-up proportional to the well area. To ensure that the Cre activity comparisons were made in a linear range, a standard curve was constructed varying the concentration of Cre-producing plasmid, measuring the output green/red ratio. Reconstituted Cre activity was calculated against this standard curve. All assays used a minimum of four samples. All assays utilized a Cre with an SV40 large T antigen nuclear localization signal in its N-terminus as a control.

Immunofluorescence/immunohistochemistry. Established protocols were used. Briefly, tissues were fixed in 4% paraformaldehyde overnight at 4° C., or 4 hours at room temperature, and prepared as frozen sections. Frozen sections conserve GFP fluorescence. Primary antibodies used were: guinea pig-anti-insulin and guinea pig anti-glucagon (Dako, Carpinteria, Calif.); rabbit anti-SS, guinea pig anti-PP (In vitrogen, Carlsbad, Calif.); rabbit anti-amylase, biotinylated Dolichos biflorus agglutinin (DBA, Sigma, St Louis, Mo.). Secondary antibodies used were: Cy3-conjugated donkey anti-rabbit IgG, Cy3-conjugated donkey anti-guinea pig IgG, Cy3-conjugated streptavidin (Jackson Immunoresearch, West Grove, Pa.). Alkaline phosphatase staining followed reported protocols (Lobe et al., 1999).

Microscopy. For whole mount YFP fluorescence, tissues were dissected and fixed in 4% paraformaldehyde (overnight, 4° C.), washed and mounted in PBS in chambers on glass slides. Samples were observed using either inverted fluorescence microscope (for regular observations) or confocal microscopy (for high quality pictures). Confocal imaging is also utilized to observe immunofluorescence-stained samples. Typically, 0.4 μm optical z-sections were taken for thick samples. A maximum of 2 adjacent optical sections were stacked and projected to produce a high quality picture for each figure.

### Example 2

### Results

Creating inactive Cre fragments for reconstitution. Reconstituting Cre activity from two inactive peptide fragments requires a pair of interacting protein motifs to bring Cre fragments to proximity for refolding. Additionally, Cre need to be cleaved at a specific site so that Cre fragments will be inactive, yet are able to reassemble into an active molecule when brought together.

The inventors considered several criteria in choosing interacting protein motifs to assist in Cre reconstitution, including high affinity, high specificity, and lack of dominant-negative effects in living cells. The reported pair of anti-parallel, heterodimer leucine zipper-forming peptides (FIGS. 1A-B, named as N— and C-peptide) fit this profile (Ghosh, 2000; Groves et al., 1998). These peptides were shown to effectively assist protein folding both in vitro and in vivo with no detectable dominant negative effects in living animals (Chelur and Chalfie, 2007; Zhang et al., 2004).

To choose the best point to separate Cre into two portions, the inventors examined the Cre three-dimensional structure for the residues and secondary structures that are crucial for its activity (Casanova et al., 2003; Gilbertson, 2003; Guo et al., 1997). The inventors choose to cleave Cre in between amino acid residues G190-G191 (FIGS. 1A-B). The flexibility of the peptide bond between glycine and other amino acid residues is more likely to tolerate addition of extra peptides without disrupting the secondary and tertiary structure of Cre. In addition, these two glycine residues are localized between two β-sheets and are expected to point away from the DNA elements during recombination (Guo et al., 1997). Therefore, connecting leucine zippers with each half of the Cre protein at this position is expected to minimally interfere with Cre function.

The inventors derived three half Cre molecules, nCre, cCre, and nlcCre, for Cre reconstitution (FIG. 1A). nCre was obtained by fusing the N-peptide to the N-terminal half Cre. The inventors included the SV40 large T antigen NLS in the N-terminus of this molecule. cCre was obtained by fusing the C-peptide to the C-terminal half Cre (FIG. 1B). nlcCre also contains a SV40 large T antigen NLS. Otherwise, it is identical to cCre (FIGS. 1A-B). The presence of an NLS in both N— and C-terminal half Cre molecules respectively is likely to restrict both molecules in the nucleus and allow for efficient interaction.

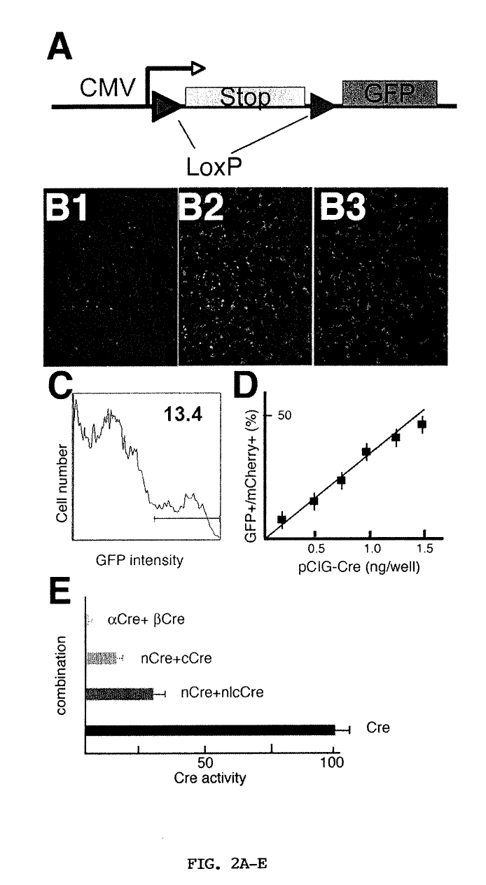

The substrate for testing reconstituted Cre activity was a reporter plasmid that produces GFP in a Cre-dependent manner (FIG. 2A). The reporter plasmid was transfected into HEK293 cells in large excess (see Materials and Methods), together with Cre-producing plasmids. An mCherry-producing plasmid (Shu et al., 2006) was co-transfected as a control for cell transfection efficiency, with the green/red fluorescence ratio providing an index for Cre activity. When 0.1 to 0.5 ng Cre-producing plasmid was used per well (12-well dishes), the green/red fluorescence ratio vs. Cre was linearly correlated (FIGS. 2B-D).

The above-described assay revealed that a combination of nCre and cCre reconstituted 12.1±2.6% recombinase activity of intact Cre molecule, whereas nCre and nlcCre recovers 27.2±3.7% Cre activity (FIG. 2E). These results demonstrate the feasibility of reconstituting significant Cre activity in cell culture. Unassisted (i.e., not aided by fused protein interaction motifs) Cre reconstitution produced a much lower level of Cre activity. Cre fragments identical to those in nCre or cCre were expressed in tissue culture, but co-expression of these fragments generated less than 0.5% of Cre activity (data not shown). Similarly, when the inventors tested the Cre fragments α5 and β1 (reported in Casanova et al., 2003), which also lack protein interaction motifs but have several overlapping residues, less than 2% Cre activity was recovered (FIG. 2E). These results demonstrate the importance of interacting leucine zippers for increased Cre reconstitution.

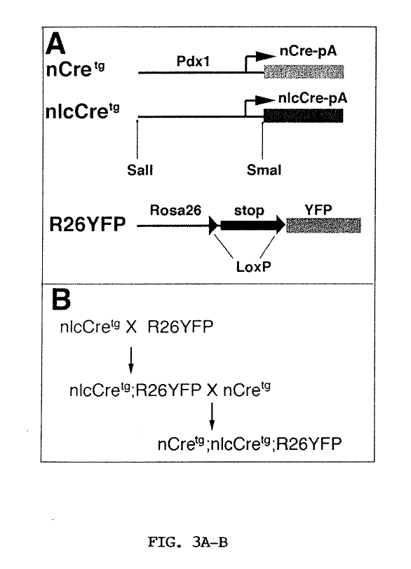

Cre activity reconstitution in transgenic mouse cells. To determine whether assisted Cre reconstitution would work in vivo, the inventors used a Pdx1 promoter to drive nCre and nlcCre expression in transgenic mice (Pdx1-nCre and Pdx1-nlcCre). The Pdx1 promoter is well-characterized, with expression restricted to all cells in the pancreas, as well as posterior foregut cells of the duodenum and antral stomach (Offield et al., 1996; Wu et al., 1997). Four Pdx1-nCre (nCretg1-4) and six Pdx1-nlcCre (nlcCre tg1-6) independent transgenic mouse lines were derived (FIG. 3A).

The inventors first crossed the nlcCre tg1 line with all four nCre tg lines to determine whether Cre activity could be reconstituted, using the Z/AP reporter allele's Cre-dependent activation of alkaline phosphatase (Lobe et al., 1999). Two lines, nCre tg1 and nCre tg3, when combined with nlcCretg1, showed AP activity in about 5% of pancreatic cells in newborn animals (data not shown). The nCre tg2 and nCre tg4 lines showed no detectable recombination when combined with nlcCretg1 and Z/AP. Semi-quantitative RT-PCR verified that these two latter lines express nearly undetectable levels of nCre mRNA (data not shown), and were sacrificed. As expected, neither nlcCre tg nor nCre tg alone could induce Z/AP recombination (data not shown).

The inventors next utilized nCre tg1 to determine which of the six nlcCre tg transgenic lines gave the highest recombination efficiency (FIG. 3B). The inventors switched to the R26YFP mouse line for this experiment because of the convenience of observing YFP fluorescence as a reporter for Cre activity (FIGS. 3A-B). Neonatal nCre tg1;nlcCre tg2;R26YFP and nCre tg1;nlcCre tg5; R26YFP animals had 31.2±4.1 (n=4) and 22.6±2.9% (n=3) pancreatic cells recombined respectively, whereas, nlcCre tg1, nlcCre tg3, nlcCre tg4, and nlcCre tg6 mouse lines showed 3-17% pancreatic cells recombined (data not shown). Our subsequent characterization used the nCre tg1 and nlcCre tg2 transgenic lines.

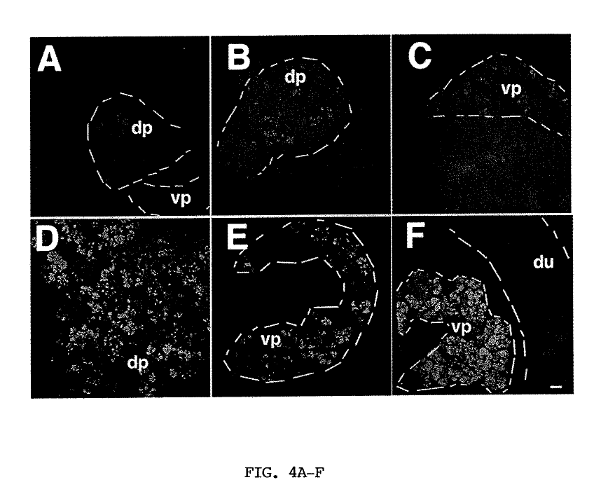

Cre activity is detected in early embryonic stages. The inventors next assessed whether there was a time dependency to the reconstitution of Cre activity as compared to the activity of the Pdx1 promoter, by assessing YFP expression in nCre tg1;nlcCre tg2;R26YFP pancreata at several stages of embryogenesis. Robust YFP expression in the pancreatic region was observed at E13.0 (8.1±3.1% of all pancreatic cells counted in 4 pancreatic buds, FIGS. 4A-C), but not at E11.5 (data not shown). The percentage of labeled cells increased gradually during embryogenesis, so that at E15.5 and E17.5, about 16.4±2.9% or 26.2±3.7% (n=4), respectively, of pancreatic cells expressed YFP (FIGS. 4D-E and data not shown). From birth to 2-month-old adults, the overall percentage of YFP+ pancreatic cells remained relatively stable (data not shown), consistent with the idea that the bulk of the pancreatic mass comprises exocrine tissues that express only a low level of Pdx1, and as such might not reach the Cre threshold for recombination of the reporter allele.

The presence of two reporter alleles substantially increases cell-labeling efficiency. One potential application for this Cre reconstitution approach is to mark and study the lineage of progenitor/stem cells that express two protein markers. It is possible that a higher percentage of cell labeling would be observed in the presence of two reporter alleles. The inventors therefore analyzed nCre tg1;nlcCre tg2;R26YFP/R26YFP embryos. Surprisingly, 63.2±5.4% (n=4) of pancreatic cells express YFP at E17.5, more than double that of the nCre tg1;nlcCre tg2;R26YFP littermates (26.5±4.9%. n=3). This result demonstrates that the presence of two floxed reporter alleles substantially increases the chance of introducing DNA recombination (in the presence of a given amount of Cre), such that more complete lineage labeling would be obtained in the presence of two reporter alleles. At present, the inventors do not understand why this increased recombination occur with the presence of two floxed alleles and it remains to be seen whether this same result holds for the other reporter alleles that are commonly in use in lineage tracing experiments in vivo.

Reconstituted Cre induces recombination in all pancreatic cell types. Effective application of Cre reconstitution requires Cre to be restored in a cell context-independent manner. The inventors therefore examined whether all pancreatic cell types could be labeled with Cre reporter expression in neonatal and adult nCre tg1;nlcCre tg2;R26YFP pancreata. The vertebrate pancreas contains two exocrine cell types, the pancreatic duct and acinar cells, and four major endocrine cell types, α, β, δ, and PP cells. The pancreatic duct cells can be recognized by their specific expression of an epitope that binds to the DBA lectin, whereas the acinar cells, α, β, δ, and PP cells can be recognized by their expression of amylase (amy), glucagon (glc), insulin (ins), somatostatin (SS), and pancreatic polypeptide (PP) respectively. The acinar cells are derived from pancreatic progenitors that continuously express high levels of Pdx1 (Gu et al., 2002). In differentiated acinar cells, a low level of Pdx1 expression is maintained (Wu et al., 1997). The β and δ cells are also derived from Pdx1+ progenitors and they maintain a high level of Pdx1 expression throughout life. On the contrary, the pancreatic duct, α, and PP cells only transiently express Pdx1 during their differentiation. If nCre and nlcCre can reassemble in a cell type independent fashion, the inventors expect that all pancreatic cell types can be labeled with Cre reporter expression, and a larger proportion of acinar, β, and δ cells should be labeled, than that of duct, α, and PP cells.

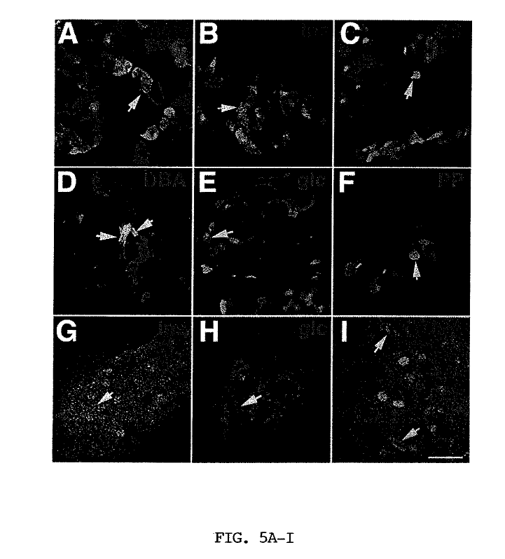

Indeed, 28.1±4.8%, 32.5±3.7%, and 21.7±6.1% acinar, β, and δ cells in neonatal nCre tg1;nlcCre tg2;R26YFP pancreas expressed YFP (n=4, FIGS. 5A-C). Whereas only 8.3±4.8%, 4.4±2.3%, and 12.5±3.7% duct, α, and PP cells expressed YFP at the same age (n=4, FIGS. 5D-F). Because pancreatic β cells maintain high levels of Pdx1 expression in postnatal animals, the inventors expected that Cre activity will be maintained in these cells in the nCre tg1;nlcCre tg2;R26YFP animals and the labeling index of the β cells should increase over age. Indeed, the percentage of YFP+ β cells increased to about 62% in 2 month-old pancreata (FIG. 5G). On the contrary, the labeling indices of cells that do not express detectable levels of Pdx1 (e.g., the duct and a cells) in postnatal pancreas did not increase, even though these labeled cells were still present in two months-old pancreas (FIGS. 5H and 5I).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,684,611
U.S. Pat. No. 4,797,368
U.S. Pat. No. 4,952,500
U.S. Pat. No. 4,959,317
U.S. Pat. No. 5,139,941
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,656,610
U.S. Pat. No. 5,670,488
U.S. Pat. No. 5,702,932
U.S. Pat. No. 5,736,524
U.S. Pat. No. 5,780,448
U.S. Pat. No. 5,789,215
U.S. Pat. No. 5,945,100
U.S. Pat. No. 5,981,274
U.S. Pat. No. 5,994,624
Abremski and Hoess, J. Biol. Chem., 259:1509-1514, 1984.
Abremski and Hoess, J. Mol. Biol., 184:211-220, 1985.
Abremski et al., Cell 32:1301-1311, 1983.
Anton and Graham, J. Virol. 4600-4606, v. 69(8), 1995.
Aoki et al., Mol. Med., 5 (4):224-31, 1999.
Baichwal and Sugden, In: Gene Transfer, Kucherlapati (ed.), New York, Plenum Press, 117-148, 1986.
Barnes et al., J. Biol. Chem., 272(17):11510-11517, 1997.
Bayley et al., Plant Mol. Biol., 18:353-361, 1992.
Bhavsa et al., Genomics, 35(1):11-23, 1996.
Bradley et al., Nature 309:255-258, 1984.
Branda and Dymecki, Dev. Cell, 6:7-28, 2004.
Brandt et al., J. Cell Physiol., 88(3):255-275, 1976.
Brinster et al., Proc. Natl. Acad. Sci. USA, 82:4438-4442, 1985.
Brunskill et al., Dev. Biol. 235, 507-520, 2001.
Casanova et al., Genesis, 37:25-29, 2003.
Case et al., Ann. NY Acad. Sci., 1044:183-200, 2005.
Chang et al., Hepatology, 14:134A, 1991.
Chelur and Chalfie, Proc. Natl. Acad. Sci. USA, 104:2283-2288, 2007.
Chen and Okayama, Mol. Cell Biol., 7(8):2745-2752, 1987.
Civin et al., J. Immunol., 133:157-165, 1984.
Clark et al., Hum. Gene Ther., 6(10):1329-1341, 1995.
Coffin, Retroviridae and Their Replication. In: Virology, Fields et al., eds., Raven Press, New York, pp. 1437-1500, 1990.
Couch et al., Am. Rev. Resp. Dis., 88:394-403, 1963.
Coupar et al., Gene, 68:1-10, 1988.
Engelmann et al., J. Mol. Cell. Cardiol. 25, 197-213, 1993.
EPO 0 273 085
Evans et al., (1981) Nature 292:154-156
Fechheimer et al., Proc. Nat'l Acad. Sci. USA, 84:8463-8467, 1987.
Flotte et al., Am. J. Respir. Cell. Mol. Biol., 7(3):349-356, 1992.
Flotte et al., Gene Ther., 2(1):29-37, 1995.
Flotte et al., Proc. Nat'l Acad. Sci. USA, 90(22):10613-7, 1993.
Fraley et al., Proc. Nat'l Acad. Sci. USA, 76:3348-3352, 1979.
Franz W M, et al., Cardioscience, 5(4):235-43, 1994.
Frederiksen et al., Neuron., 1:439-448, 1988.
Friedmann, Science, 244:1275-1281, 1989.
Ghosh and Bachhawat, In: Liver Diseases, Targeted Diagnosis and Therapy Using Specific Receptors and Ligands. Wu et al., eds., Marcel Dekker, New York, pp. 87-104, 1991.
Ghosh et al., J. Am. Chem. Soc., 122:5658-5659, 2000.
Ghosh-Choudhury et al., EMBO J., 6:1733-1739, 1987.
Gilbertson, Trends Biotechnol., 21:550-555, 2003.
Gomez-Foix et al., J. Biol. Chem., 267:25129-25134, 1992.
Gopal, Mol. Cell Biol., 5:1188-1190, 1985.
Gopal-Srivastava et al., J. Mol. Cell. Biol. 15(12):7081-90, 1995.
Gordon et al., Proc. Natl. Acad. Sci. USA, 77:7380-7384, 1980.
Gossler et al., Proc. Nat'l Acad. Sci. USA 83: 9065-9069, 1986.
Graham and Prevec, Biotechnology, 20:363-390, 1992.
Graham and Prevec, In: Methods in Molecular Biology. Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Gray et al., Science, 306:2255-2257, 2004.
Groves et al., FEBS Lett., 433:223-227, 1998.
Grunhaus et al., Seminar in Virology, 200(2):535-546, 1992.
Gu et al., Cell 72, 309-324, February 1993.
Gu et al., Development, 129:2447-2457, 2002.
Gu et al., Development, 131:165-79, 2004.
Gu et al., Mech. Dev., 120:35-43, 2003.
Gunther et al., Mol. Cell Biol., 15:2490-2499, 1995.
Guo et al., Nature, 389:40-46, 1997.
Harland and Weintraub, J. Cell Biol., 101:1094-1099, 1985.
Hermonat and Muzycska, Proc. Nat'l Acad. Sci. USA, 81:6466-6470, 1984.
Herrera, Development, 127:2317-2322, 2000.
Hersdorffer et al., DNA Cell Biol., 9:713-723, 1990.
Herz and Gerard, Proc. Nat'l Acad. Sci. USA, 90:2812-2816, 1993.
Hoess et al., Proc. Nat'l Acad. Sci. USA, 79:3398, 1982.
Hogan et al., In: Manipulating the Mouse Embryo. A Laboratory Manual, 2nd ed., Cold Spring Harbor Laboratory Press, 1994.
Horwich et al, Virol., 64:642-650, 1990.
Howell et al., Ann. NY Acad. Sci., 996:158-73, 2003.
Hunte et al., Eur. J. Immunol., 28:3850-3856, 1998.
Ishikawa et al., Faseb J., 20:950-952, 2006.
Ito et al., Nuc. Acid Res. 10:1755. 1982.
Jaenich, Proc. Natl. Acad. Sci. USA, 73:1260-1264, 1986.
Jaenisch, Science 240:1468-1474, 1988.
Jahn et al., J. Cell Science, 109:397-405, 1996.
Jahner et al., Nature 298:623-628, 1982.
Jahner et al., Proc. Nat'l Acad. Sci. USA 82:6927-6931, 1985.
Johnsson and Varshavsky, Proc. Natl. Acad. Sci. USA, 91:10340-10344, 1994.
Jones and Shenk, Cell, 13:181-188, 1978.
Jullien et al., Nucleic Acids Res., 31:e131, 2003.
Kaneda et al., Science, 243:375-378, 1989.
Kaplitt et al., Nat Genet., 8(2):148-54, 1994.
Karlsson et al., EMBO J., 5:2377-2385, 1986.
Kasahara et al., Science, 266(5189):1373-6, 1994.
Kato et al., J. Biol. Chem., 266:3361-3364, 1991.
Kawaguchi et al., Nat. Genet., 32:128-134, 2002.
Kawamoto et al., FEBS Letters 470 (2000) 263-268.
Kellendonk et al., Nucleic Acids Res., 24:1404-1411, 1996.
Kelly et al., J. Cell. Biol., 129(2):383-96, 1995.
Kiel et al., Cell, 121:1109-1121, 2005.
Kim et al., Cell, 121:823-835, 2005.
Kimes and Brandt, Exp. Cell Res., 98(2):367-381, 1976.
Kimura et al., Dev. Growth Differ. 39(3):257-65, 1997.
Klein et al., Nature, 327:70-73, 1987.
Kline et al., J. C. V. Electrophys., 4(6), 642-660, 1993.
Klug et al., J. Clin. Invest. 98, 216-224, 1996.
Kotin et al., Proc. Nat'l Acad. Sci. USA, March 1990;87(6):2211-5, 1990.
LaFace et al., Virology, 162(2):483-486, 1988.
Lakso et al., Proc. Natl. Acad. Sci. USA, 89:6232-6236, 1992.
LaPointe et al., Hypertension 27(3 Pt 2):715-22, 1996.
Laughlin et al., J. Virol., 60(2):515-524, 1986.
Le et al., Nucleic Acids Res., 27:4703-4709, 1999.
Le Gal La Salle et al., Science, 259:988-990, 1993.
Lebkowski et al., Mol. Cell. Biol., 8(10):3988-3996, 1988.
LePage and Conlon, Methods Mol. Med., 129:41-67, 2006.
Levrero et al., Gene, 101:195-202, 1991.
Lewandoski, Nat. Rev. Genet., 2:743-755, 2001.
Lien et al., Development, 126(1):75-84, 1999.
Liu et al., Genes Dev., 2:528-541, 1988.
Lobe et al., Dev. Biol., 208:281-292, 1999.
Macejak and Sarnow, Nature, 353:90-94, 1991.
Magliery et al., J. Am. Chem. Soc., 127:146-157, 2005.
Magliery et al., J. Am. Chem. Soc., 127:146-157, 2005.
Mann et al., Cell, 33:153-159, 1983.
Markowitz et al., J. Virol., 62:1120-1124, 1988.
McCarty et al., J. Virol., 65(6):2936-45, 1991.
McLaughlin et al., J. Virol., 62(6):1963-1973, 1988.
Metzger and Chambon, Methods, 24:71-80, 2001.
Miller et al., Am. J. Clin. Oncol., 15(3):216-221, 1992.
Morgan et al., Devel. Biol. 162, 486-498, 1994.
Moss et al., Biol. Chem., 271(49):31688-94, 1996.
Muzyczka, Curr. Topics Microbiol. Immunol., 158:97-129, 1992.
Nicolas and Rubinstein, In: Vectors. A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Offield et al., Development, 122:983-995, 1996.
Ogilvie et al., Science 214:270, 1981
Ohi et al., Gene, 89(2):279-282, 1990.
Omirulleh et al., Plant Mol. Biol., 21(3):415-28, 1993.
Paskind et al., Virology, 67:242-248, 1975.
PCT Application WO 94/09699
PCT Application WO 95/06128
PCT Application WO/93/19172
PCT Application WO/94/09699
Pelletier and Sonenberg, Nature, 334(6180):320-325, 1988.
Pelletier et al., Nat. Biotechnol., 17:683-690, 1999.
Pelletier et al., Proc. Natl. Acad. Sci. USA, 95:12141-12146, 1998.
Perales et al., Proc. Nat'l Acad. Sci. USA, 91:4086-4090, 1994.
Porret et al., Methods Mol. Biol., 337:185-205, 2006.
Potrykus et al., Mol. Gen. Genet., 199(2):169-77, 1985.
Potter et al., Proc. Natl. Acad. Sci. USA, 81:7161-7165, 1984.
Qin et al., Proc. Natl. Acad. Sci. USA, 91:1706-1710, 1994.
Racher et al., Biotechnology Techniques, 9:169-174, 1995.
Radhakrishnan and Srivastava, Plant Cell Rep., 23:721-726, 2005.
Ragot et al., Nature, 361:647-650, 1993.
Rajewsky et al., J. Clin. Invest., 98:600-603, 1996.
Remy and Michnick, Methods Mol. Biol., 261:411-426, 2004.
Renan, Radiother. Oncol., 19:197-218, 1990.
Rich et al., Hum. Gene Ther., 4:461-476, 1993.
Richards and Vithayathil, J. Biol. Chem., 234:1459-1465, 1959.
Ridgeway, In: Vectors. A survey of molecular cloning vectors and their uses, Stoneham: Butterworth, pp. 467-492, 1988.
Rippe et al., Mol. Cell Biol., 10:689-695, 1990.
Ritchie, J. Biol. Chem. 271(41):25485-25491, 1996.
Robertson et al., Nature 322:445-448, 1986
Rosenfeld et al., Cell, 68:143-155, 1992.
Rosenfeld et al., Science, 252:431-434, 1991.
Rossant and Nagy, Nat. Med. 1:592-594, 1995.
Rossi et al., Proc. Natl. Acad. Sci. USA, 94:8405-8410, 1997.
Roux et al., Proc. Natl. Acad. Sci. USA, 86:9079-9083, 1989.
Samulski et al., EMBO J., 10:3941-3950, 1991.
Samulski et al., J. Virol, 63:3822-3828, 1989.
Saue and Henderson, Proc. Natl. Acad. Sci. USA, 85:5166-5170, 1988.
Sauer, Endocrine, 19:221-228, 2002.
Sauer, Methods Enzymol., 225:890-900, 1993.
Sauer, Methods of Enzymology, 225, 890-900, 1993.
Sauer, Mol. Cell Biol., 7:2087-2096, 1987.
Shelling and Smith, Gene Therapy, 1:165-169, 1994.
Shiba and Schimmel, Proc. Natl. Acad. Sci. USA, 89:1880-1884, 1992.
Shimshek et al., Genesis, 32:19-26, 2002.
Shimshek et al., Genesis, 32:19-26, 2002.
Shu et al., Biochemistry, 45:9639-9647, 2006.
Sohal et al., Circ. Res. 89:20-25, 2001.
Srinivas et al., BMC Dev. Biol., 1:4, 2001.
Steinhelper et al., Am. J. Physiol. 259 (6 Pt 2), H1826-34, 1990.
Stewart et al., EMBO J. 6:383-388, 1987.
Temin, In: Gene Transfer, Kucherlapati (ed.), New York: Plenum Press, pp. 149-188, 1986.
Theodosis et al., Psychoneuroendocrinology, 19:455-462, 1994.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Tur-Kaspa et al., Mol. Cell Biol., 6:716-718, 1986.
Uchida et al., Proc. Natl. Acad. Sci. USA, 97:14720-14725, 2000.
Van der Putten et al., Proc. Nat'l Acad. Sci. USA 82:6148-6152, 1985.
Wagner et al., Proc. Nat'l Acad. Sci. USA 87(9):3410-3414, 1990.
Walsh et al., J. Clin. Invest, 94:1440-1448, 1994.
Wei et al., Gene Therapy, 1:261-268, 1994.
Wiemann et al., Genome Res., 11:422-435, 2001.
Wilson et al., Nat. Methods, 1:255-262, 2004.
Wong et al., Gene, 10:87-94, 1980.
Wu and Wu, J. Biol. Chem., 262:4429-4432, 1987.
Wu and Wu, Adv. Drug Delivery Rev., 12:159-167, 1993.
Wu and Wu, Biochemistry, 27:887-892, 1988.
Xu et al., Dev. Biol., 297:340-349, 2006.
Yamauchi-Takihara, et. al., Proc. Natl. Acad. Sci. USA, 86(10):3504-3508, 1989.
Yang et al., Proc Natl. Acad. Sci. USA, 87:9568-9572, 1990.
Yang et al., Proc. Natl. Acad. Sci. USA, 91:4407-4411, 1994.
Yoder et al., Bio/Technology 12:263-268, 1994.
Yu et al., Circ. Res. 79:691-697, 1996.
Zhang et al., Cell, 119:137-144, 2004.
Zhou et al., Exp. Hematol, 21:928-933, 1993.
Zhou et al., J. Exp. Med., 179:1867-1875, 1994.
Ziober and Kramer, J. Bio. Chem., 271(37):22915-22, 1996.

What is claimed is:

1. An active Cre recombinase comprising:
(i) an N-terminal segment of Cre fused to a first leucine zipper peptide, wherein said first leucine zipper peptide is fused to the C-terminal end of said N-terminal segment; and
(ii) a C-terminal segment of Cre fused to a second leucine zipper peptide, wherein said second leucine zipper peptide is fused to the N-terminal end of said C-terminal segment.

2. The active Cre recombinase of claim 1, wherein said first and/or second leucine zipper peptide is between 20 and 40 residues in length.

3. The active Cre recombinase of claim 2, wherein said first leucine zipper comprises the sequence GALKKELQANKKELAQLKWELQALKKELAQ (SEQ ID NO:1).

4. The active Cre recombinase of claim 2, wherein said second leucine zipper comprises the sequence EQLEKKLQALEKKLAQLEWKNQALEKKLAQG (SEQ ID NO:2).

5. The active Cre recombinase of claim 1, wherein said N-terminal segment comprises residue 1 to about residue 190 of native Cre recombinase.

6. The active Cre recombinase of claim 1, wherein said C-terminal segment comprises from about residue 191 to residue 320 of native Cre recombinase.

7. The active Cre recombinase of claim 1, wherein said N-terminal and/or said C-terminal segments further comprise a nuclear localization signal.

8. The active Cre recombinase of claim 7, wherein said nuclear localization signal is an SV40 nuclear localization signal.

9. The active Cre recombinase of claim 7, wherein said nuclear localization signal is located at the N-terminus of said N-terminal segment.

10. The active Cre recombinase of claim 7, wherein said nuclear localization signal is located at the N-terminus of said second leucine zipper peptide.

11. An N-terminal segment of Cre fused to a leucine zipper peptide, wherein said leucine zipper peptide is fused to the C-terminal end of said N-terminal segment.

12. The N-terminal segment of claim 11, wherein said N-terminal segment further comprises a nuclear localization signal.

13. A C-terminal segment of Cre fused to a leucine zipper peptide, wherein said leucine zipper peptide is fused to the N-terminal end of said C-terminal segment.

14. The C-terminal segment of claim 13, wherein said C-terminal segment further comprises a nuclear localization signal.

15. An expression construct encoding (i) a fusion protein comprising an N-terminal segment of Cre fused to a leucine zipper peptide, wherein said leucine zipper peptide is fused to the C-terminal end of said N-terminal segment, and (ii) a promoter.

16. The expression construct of claim 15, wherein said fusion protein further comprises a nuclear localization signal.

17. An expression construct encoding a (i) a fusion protein comprising a C-terminal segment of Cre fused to a leucine zipper peptide, wherein said leucine zipper peptide is fused to the N-terminal end of said C-terminal segment, and (ii) a promoter.

18. The expression construct of claim 17, wherein said fusion protein further comprises a nuclear localization signal.

19. A kit comprising, in separate containers:
(a) an expression construct encoding (i) a first fusion protein comprising an N-terminal segment of Cre fused to a first leucine zipper peptide, wherein said first leucine zipper peptide is fused to the C-terminal end of said N-terminal segment, and (ii) a promoter that directs expression of said first fusion protein; and
(b) an expression construct encoding (i) a second fusion protein comprising a C-terminal segment of Cre fused to a second leucine zipper peptide, wherein said second leucine zipper peptide is fused to the N-terminal end of said C-terminal segment, and (ii) a promoter that directs expression of said second fusion protein.

20. The kit of claim 19, wherein said first and/or second fusion proteins further comprise a nuclear localization signal.

21. A method for introducing cre recombinase activity into a cell lacking Cre recombinase activity comprising:
(a) a first expression cassette encoding (i) a first fusion protein comprising an N-terminal segment of Cre fused to a first leucine zipper peptide, wherein said first leucine zipper peptide is fused to the C-terminal end of said N-terminal segment, and (ii) a promoter that directs expression of said first fusion protein; and
(b) a second expression cassette encoding (i) a first fusion protein comprising a C-terminal segment of Cre fused to a second leucine zipper peptide, wherein said second leucine zipper peptide is fused to the N-terminal end of said C-terminal segment and (ii) a promoter that directs expression of said second fusion protein.

22. The method of claim 21, wherein said first and/or second leucine zipper peptide is between 20 and 40 residues in length.

23. The method of claim 22, wherein said first leucine zipper comprises the sequence GALKKELQANKKELAQLKWELQALKKELAQ (SEQ ID NO:1).

24. The method of claim 22, wherein said second leucine zipper comprises the sequence EQLEKKLQALEKKLAQLEWKNQALEKKLAQG (SEQ ID NO:2).

25. The method of claim 21, wherein said N-terminal segment comprises residue 1 to about residue 190 of native Cre recombinase.

26. The method of claim 21, wherein said C-terminal segment comprises from about residue 191 to residue 320 of native Cre recombinase.

27. The method of claim 21, wherein said first and second fusion proteins further comprise a nuclear localization signal.

28. The method of claim 27, wherein said nuclear localization signal is an SV40 nuclear localization signal.

29. The method of claim 27, wherein said nuclear localization signal is located at the N-terminus of said N-terminal segment.

30. The method of claim 27, wherein said nuclear localization signal is located a the N-terminus of said second leucine zipper peptide.

31. The method of claim 21, wherein introducing comprises non-viral transfer.

32. The method of claim 31, wherein non-viral transfer comprises contacting a lipid vehicle comprising said expression cassettes with said cell.

33. The method of claim 21, wherein introducing comprises viral transfer.

34. The method of claim 33, wherein viral transfer comprises contacting said cell with
a first replication deficient vector encoding said first expression cassette; and
a second replication deficient vector encoding said second first expression cassette.

35. The method of claim 33, wherein viral transfer comprises contacting said cell with a replication deficient viral vector encoding said first expression cassette and said second first expression cassette.

36. The method of claim 21, wherein introducing comprises pronuclear injection.

37. The method of claim 21, wherein said cell is stem cell.

38. The method of claim 37, wherein said first promoter is a promoter for a first stem cell marker.

39. The method of claim 37, wherein said second promoter is a promoter for a second stem cell marker.

40. The method of claim 37, further comprising introducing a marker segment into said cell, said marker segment comprising an expression cassette comprising two loxP sites and a detectable or selectable marker gene under the control of a third promoter active in said cell, said expression cassette flanked by said loxP sites.

41. The method of claim 21, wherein said cell is a zygote.

42. The method of claim 21, wherein said cell is a pluripotent cell.

43. The method of claim 21, wherein said cell is a totipotent cell.

44. The method of claim 21, wherein said cell is a gene therapy target cell.


SEQUENCE LISTING (6 sequences)

SEQ ID NO: 1 - protein, 30 aa, Artificial
  Synthetic peptide
GALKKELQANKKELAQLKWELQALKKELAQ

SEQ ID NO: 2 - protein, 31 aa, Artificial
  Synthetic peptide
EQLEKKLQALEKKLAQLEWKNQALEKKLAQG

SEQ ID NO: 3 - protein, 8 aa, Artificial
  Synthetic peptide
PPKKKRKV

SEQ ID NO: 4 - DNA, 34 bp, Artificial
  Synthetic primer
ataacttcgtataatgtatgctatacgaagttat

SEQ ID NO: 5 - DNA, 34 bp, Artificial
  Synthetic primer
acaacttcgtataatgtatgctatacgaagttat

SEQ ID NO: 6 - DNA, 34 bp, Artificial
  Synthetic primer
ataacttcgtataatgtatactatacgaagttat